(19) United States
(12) Patent Application Publication
(10) Pub. No.: US 2011/0190741 A1
(43) Pub. Date: Aug. 4, 2011

(54) Coupling of an eye to a laser device

(76) Inventors: Thomas Deisinger, Cadolzburg (DE); Olaf Kittelmann, Berlin (DE); Jing Li, Erlangen (DE); Gerhard Robl, Stein (DE); Klaus Vogler, Eckental (DE); Bend Zerl, Uttenreuth (DE)

(21) Appl. No.: 13/035,284

(22) Filed: Feb. 25, 2011

(30) Foreign Application Priority Data
Aug. 25, 2008 (EP) PCT/EP2008/006962

(51) Int. Cl. A61B 18/20

(52) U.S. Cl. 606/5

(57) ABSTRACT

According to an exemplary embodiment, a process for coupling a mechanical interface unit to a suction-ring unit retained on an eye by suction force includes a step of relative approximating of the interface unit to the suction-ring unit in an axial direction as far as a first, predetermined relative position of the two components, which is preferentially detected by suitable sensorics. When the first relative position is attained, the evacuation is begun of a suction chamber formed between the interface unit, the suction-ring unit and the surface of the eye, whereby the partial vacuum generated establishes in the suction chamber a contact between the surface of the eye and an applanation plate retained on the interface unit, or enlarges a region of existing contact. The aspirating of the eye onto the applanation plate prevents compression loads and shear loads on the eye such as normally cannot be avoided when impressing the plate onto the eye.

This application claims priority to internationally filed application PCT/EP2008/006962 filed on Aug. 25, 2008.

The invention is concerned generally with the establishment of a mechanical coupling between a biological tissue and a laser device, the laser radiation of which is utilised for the purpose of treating the tissue. In particular, the invention is concerned with the coupling of a laser device to an eye, in particular a human eye, in order to introduce one or more incisions into the eye with the laser radiation.

For a targeted action of the laser radiation a precise localisation of the beam focus relative to the tissue to be treated is indispensable. In particular for the generation of incisions in the tissue, frequently a relatively small beam focus is striven for, in order to keep the thickness of the incision small. The positioning of the beam focus then also has to be correspondingly precise. This holds, in particular, for the introduction of incisions into ocular tissue, such as occur, for example, within the scope of so-called fs LASIK. LASIK stands for laser in-situ keratomileusis and designates a technique for treating eyesight defects, in which firstly a covering disc (in specialist circles normally designated as a flap) is cut out of the anterior region of the cornea, which in part still remains connected to the cornea so that it can be folded aside for a subsequent ablation of underlying corneal tissue by means of laser radiation. After implementation of the ablation (resection of tissue), the flap is folded back, and a relatively rapid healing takes place, with the corneal surface largely undamaged.

For the production of the flap, a previously common technique uses a mechanical planer (microkeratome) which cuts into the cornea from the side with a rapidly oscillating cutting blade. Work has also been proceeding for some time on systems that permit generation of a flap by means of focused laser radiation with pulse durations within the femtosecond range. Hence the name femtosecond LASIK or fs LASIK. The radiation in this case is focused beneath the anterior surface of the cornea in the interior of the tissue, and the focal points are positioned in the desired surface in such a way that, as a result, a flap is cut out of the cornea.

But tissue incisions in the eye are needed not only in fs LASIK but also in other indications, for instance in keratoplasty (e.g. anterior or posterior lamellar keratoplasty, penetrating keratoplasty in corneal graftings), in fs lenticle extraction for the purpose of refraction correction, in the cutting of intercorneal ring segments for the purpose of stabilising keratoconus and protrusion of the cornea (e.g. for the insertion of intacs, i.e. small implanted ring segments for biomechanical stabilisation of the cornea), in cataract incisions, in presbyopia incisions in the crystalline lens, in intrastromal inlays, in keratotomy for astigmatisms, in corneal resection and such like.

In the state of the art it is known (see e.g. U.S. Pat. No. 5,549,632, WO 03/002008 A1) in the case of eye-laser devices to impress a plane-parallel applanation lens onto the cornea. As a result of the impressing of the applanation lens, the eye is deformed and conforms two-dimensionally to the underside of the applanation lens facing towards the eye. The beam focus of the laser radiation is referenced in the z-direction in relation to the applanation lens (the z-direction in this connection means the longitudinal direction of the beam). By virtue of the abutting of the eye against the applanation lens, there is a fixed z-reference between eye and lens, permitting a precise z-positioning of the beam focus in arbitrary regions in the cornea or in other tissue structures deep within the eye.

Besides plane-parallel applanation lenses, lenses (or generally contact glasses) have also become known in the state of the art having spherically, aspherically or otherwise curved surfaces, which equally enable a z-referencing. By virtue of suitably concave design of the underside of the lens facing towards the eye, the deformation of the eye in the course of mounting the lens can be reduced. This is advantageous to the extent that the intraocular pressure does not increase so much as in the case of an impressed applanation lens having a flat underside. However, the curved lens surfaces impair the focusability of the laser radiation.

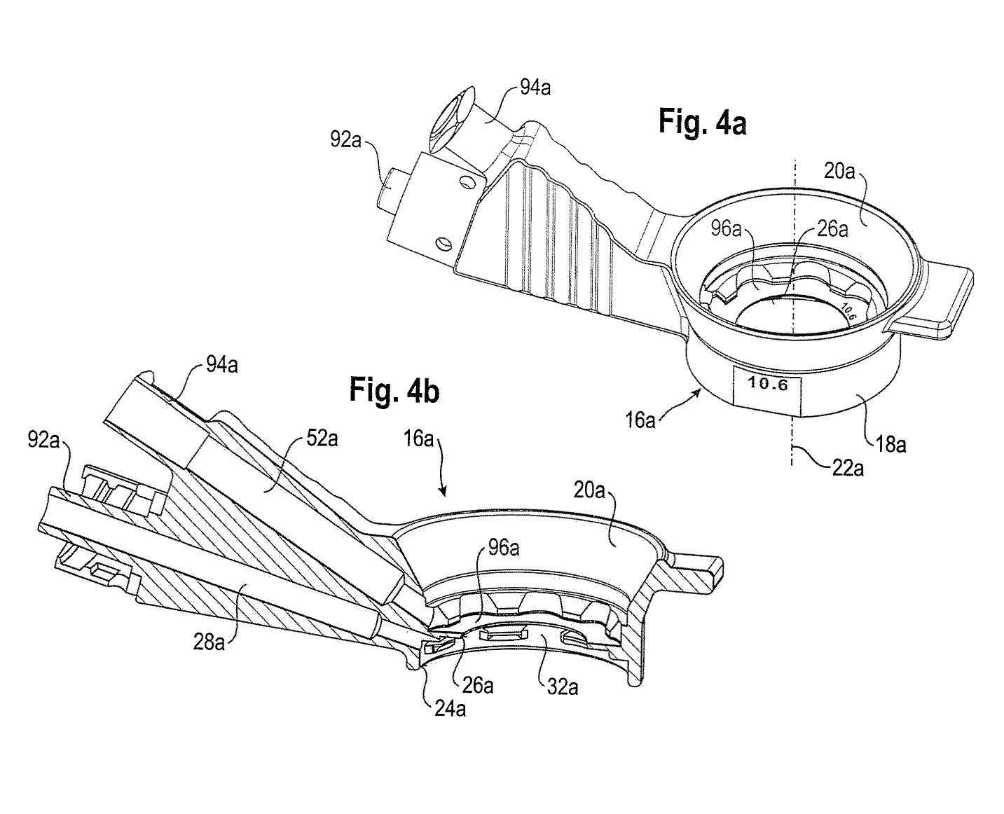

In order to keep the eye of the patient at a fixed distance from the focusing optics of the laser device, in the state of the art use is made, as a rule, of suction rings which are aspirated onto the sclera of the eye by means of partial vacuum and surround the cornea in the form of a ring. Either the applanation lens in this case is integrated into the suction ring—as shown, for example, in FIG. 4C of the aforementioned U.S. Pat. No. 5,549,632—or the applanation lens is part of a separate component which is coupled with the suction ring; see, for example, FIG. 7 of WO 03/002008 A1, where the applanation lens is permanently filled to a conical body which in the region of its cone base is constructed for coupling to the focusing optics of a laser device and at its narrow cone end can be brought into firm coupling with the suction ring by means of a separate compression forceps.

The tripartite structural design according to WO 03/002008 A1—with a suction ring, a compression forceps and a conical body bearing the applanation lens—permits a mutually independent guidance of the treatment optics of the laser device and of the eye of the patient right up close to one another. The suction ring in this case is already seated on the eye, whereas the cone is already fitted to the treatment optics. Once the treatment optics and the eye of the patient have been brought close enough to one another, the compression forceps, which establishes the mechanical coupling between the cone and the suction ring, comes into operation.

It is readily comprehensible that the mechanical fixation of the eye in relation to the treatment optics by means of the compression forceps is a critical phase of the surgical preparation. The compressive forces and shear forces acting in this process must not result in injuries to the eye of the patient or bring about an excessive and possibly dangerous increase in the intraocular pressure as a result of imprecise positioning of the mechanical components involved relative to one another. If an increased intraocular pressure occurs over a relatively long period, under certain circumstances this may result in damage to the optic nerve. Even if it is possible to position the optics more or less precisely by means of a joystick, the cone is nevertheless rigidly connected to the optics, for which reason the mechanical contact finally brought about with the eye and the associated levelling of the eye remain rigid. The forces exerted on the eye in this connection—specifically, both the forces arising during the procedure of coupling and the forces acting after applanation has taken place—are hardly predictable and may be different from patient to patient.

Overall, for numerous solutions known in the state of the art it is characteristic that an applanation lens—or, expressed more generally, a contact glass, no matter of what shape—is impressed onto the eye in the course of the coupling of the eye to the treatment optics, specifically so strongly that the eye conforms to the contact glass in the region required for the following treatment. This impressing of the contact glass onto the eye is normally associated with pressure surges which may be felt to be unpleasant by the patient. The eye is squeezed in the process and under certain circumstances may suffer damage, particularly because the tensile, compressive and shear forces arising cannot be precisely defined in advance.

The object of the invention is to enable a more considerate coupling of a body tissue, in particular an eye, to a laser device.

For the purpose of achieving this object, according to one aspect a process is provided for coupling a mechanical interface unit of a laser device to a stabilising component retained on a biological tissue by suction force, the process comprising the following steps:
relative approximating of the interface unit to the stabilising component as far as a first relative position,
in the first relative position, evacuating a suction chamber formed between the interface unit, the stabilising component and the surface of the tissue, in order thereby to establish a contact between the tissue and a tissue-abutment surface of the interface unit, or to enlarge a region of existing contact.

In the case of the solution according to the invention, the interface unit and the stabilising component are firstly approximated to one another until they attain a predetermined first relative position. This relative approximation may be effected, for example, by means of motorised drive means or manually. As soon as the first relative position has been attained, the evacuation begins of a suction chamber which is formed between the interface unit and the stabilising component but which is also, in part, bounded by the surface of the tissue. The evacuation of the suction chamber consequently ensures a mutual aspirating of the interface unit and of the stabilising component, retaining these on one another. But the partial vacuum also acts on parts of the tissue surface. In accordance with the invention, this is utilised in order to suck the tissue against a tissue-abutment surface formed by the interface unit, so that a two-dimensional conforming of the tissue to the tissue-abutment surface takes place there. In the first relative position of the interface unit and of the stabilising component, in one configuration of the invention there is not yet any contact between the tissue and the tissue-abutment surface before the evacuation of the suction chamber is begun. According to an alternative configuration, such a contact may already exist, which, however, is significantly enlarged by the evacuation of the suction chamber. The geometry of the interface unit and of the stabilising component as well as the strength of the partial vacuum generated in the suction chamber are such that, after evacuation has taken place, the tissue bears against the tissue-abutment surface in a region that is at least as large as the extent of the intended treatment region but at the start of the evacuation is still significantly smaller than this treatment region.

In particular, the evacuation of the suction chamber brings about an enlargement of the region of contact between the tissue and the tissue-abutment surface to at least one and a half times, preferentially to at least double, and most preferentially to a multiple. In the case of an approximately circular-disc-shaped region of contact between the tissue and the tissue-abutment surface—such as normally occurs, for example, in the case of an eye—the evacuation of the suction chamber should bring about an enlargement of the diameter of the region of contact by at least 30 percent, preferentially by at least 50 percent, still more preferably by at least 70 percent, and most preferably by at least 90 percent.

As distinct from known solutions in the field of laser-surgery ophthalmology, the invention does not rely solely on the levelling or other shaping of the tissue to be treated by impressing a contact element onto the tissue, but rather on the generation of a suction effect on the parts of the tissue surface in question, by which the tissue is sucked against the tissue-abutment surface. This fundamentally different mode of action permits pressure surges and squeezing phenomena, such as are unavoidable in the case of the total impressing of a contact element, to be avoided or—provided that a certain small-area contact between tissue and tissue-abutment surface is already present at the start of the evacuation—at least to be greatly reduced. Uncontrolled forces do not arise (not even temporarily) or arise at least only to an extremely slight extent. Because the suction force that is capable of being set in defined and reproducible manner via the strength of the partial vacuum acts vertically away from the surface of the tissue, no lateral shear forces arise which in the eye could easily result in cases of damage to the epithelium.

A further advantage of the solution according to the invention consists in the problem-free uncoupling of the interface unit from the stabilising component in the case of complications or in the case of a panic reaction of the patient. By aeration of the suction chamber, the coupling between interface unit and stabilising component, and hence between tissue and laser device, can be released instantaneously, i.e. without delay.

In the case of eye treatments, the internal pressure of the eye is only increased slightly, if at all, by the aspirating of the cornea onto the abutment surface of the interface unit. This allows the patient to feel the operation to be more agreeable.

According to one further development of the invention, the relative approximation of the interface unit to the stabilising component may be effected in a direction along which a further relative approximation of the interface unit and of the stabilising component beyond the first relative position is possible, in particular as far as a state of reciprocal impact. This means that the first relative approximation of the interface unit and of the stabilising component is stopped even before the two have been maximally approximated to one another. In the first relative position, in one possible embodiment a certain spacing between the interface unit and the stabilising component in the direction of the approximating movement may accordingly be present. By virtue of the subsequent evacuation of the suction chamber, this spacing may diminish, namely in such a manner that the evacuation of the suction chamber brings about a relative movement of the interface unit and of the stabilising component into a second relative position in which they are more greatly approximated to one another than in the first relative position. It is to be observed that this further relative approximation of the interface unit and of the stabilising component beyond the first relative position is brought about solely by the partial vacuum prevailing in the suction chamber and not by an external motorised or manual application of force to the interface unit or/and the stabilising component In particular, the partial vacuum in the suction chamber brings about a reciprocal attraction of the interface unit and of the stabilising component into a stop position in which further approximation is no longer possible.

The path of relative movement of the interface unit and of the stabilising component between the first and the second relative positions is expediently comparatively small and preferentially amounts to no more than 1 mm. In particular, by virtue of the evacuating of the suction chamber a further approximation is brought about of the interface unit and of the stabilising component by distinctly less than one millimetre, for example by only two or three tenths of a millimetre. In each case it is advisable that a proportion of the enlargement of the region of contact between the tissue and the tissue-abutment surface that is to be ascribed to the relative approximation of the interface unit and of the stabilising component from the first into the second relative position is small, in particular negligibly small in comparison with a proportion to be ascribed to the aspirating of the tissue onto the tissue-abutment surface. Expressed otherwise, the mere approximation of the interface unit and of the stabilising component into the second relative position should not in itself ensure a significant increase in the region of contact between tissue and tissue-abutment surface. This can be guaranteed, for example, by the path of movement between first and second relative positions amounting to only a fraction of a millimetre.

The invention is suitable, in particular, for an automated process sequence in which at least parts of the coupling procedure proceed in automated manner. For example, the attaining of the first relative position can be detected by means of sensorics, and the evacuation of the suction chamber can be brought about automatically, in particular in program-controlled manner, in response to the detection of the first relative position. Alternatively or in addition, the relative approximation of the interface unit to the stabilising component can be brought about by actuation of a motorised drive device, and the operation of the drive device can be stopped automatically, in particular in program-controlled manner, in response to a sensoric detection of the first relative position.

According to a further aspect of the invention, with a view to achieving the object stated in the introduction an apparatus is provided for cutting a tissue part of an eye by means of focused laser radiation, this apparatus being suitable, in particular, for implementing the process of the type elucidated above, and comprising the following elements:
a suction-ring unit to be placed onto the eye, with a ring axis,
a mechanical interface unit which is separate from the suction-ring unit and capable of moving along the ring axis in coupling contact with the latter, with a contact glass for shaping the surface of the eye,
pumping means for evacuating a first suction chamber bounded between the suction-ring unit, the interface unit and the surface of the eye,
sensorics for detecting a predetermined first axial relative position of the interface unit and of the suction-ring unit,
a control device, in particular a program-controlled control device, connected to the sensorics and to the pumping means, which has been set up to bring about an evacuation of the first suction chamber in response to the detection of the first relative position by the sensorics, in particular in order thereby to establish a contact between the eye and a shaping abutment surface of the contact glass, or to enlarge a region of existing contact.

By way of contact glass here, any transparent contact element is to be understood that serves for shaping contact by the surface of the eye. The word ‘glass’ is not to be understood as a reference to a particular glass material of the contact glass; instead, it is solely intended to make clear the transparency in respect of the laser radiation. Even if in many cases in practice a glass material may find application for the contact glass, it is just as conceivable to manufacture the contact glass from a plastic material. In a preferred embodiment, the contact glass is constructed as a plane-parallel applanation lens, possessing a flat lens surface both on its side facing towards the eye and also on its side facing away from the eye. Within the scope of the invention it is, of course, not excluded in principle to construct the contact glass with principal sides that are not flat.

The sensorics may include, for example, a proximity sensor which, for example, may take the form of a Hall-effect sensor, an optical sensor or a reed switch. It is also conceivable to provide an optical sensor in the manner of a light barrier, in order to detect the attaining of the predetermined first relative position of the interface unit and of the suction-ring unit. Furthermore, it is conceivable to provide a pressure sensor or force sensor which measures a counterpressure from the eye or from the suction-ring unit acting on the interface unit. In the case of a weight-compensated suspension of the treatment optics of a laser device providing the laser radiation, the sensorics may also include a switch that switches in the case of a certain displacement of the optics from a normal position, which said optics undergo when the interface unit coupled with the optics impinges on the surface of the eye or on the suction-ring unit and in this way experiences a counterpressure.

The suction-ring unit represents one example of a stabilising component in the sense of the invention; it stabilises and fixes the eye. It may form a full-perimeter first sealing surface in the form of a ring with which it is capable of being applied onto the surface of the eye for the purpose of sealing the first suction chamber, whereby the evacuation of the first suction chamber brings about an abutment of the eye against the contact glass in a region that corresponds approximately to the area surrounded by the first sealing surface. Via the diameter or the cross-sectional size of the region surrounded by the first sealing surface, the size of the region of contact between eye and contact glass can be established in this way.

In a preferred embodiment, the first sealing surface separates the first suction chamber from a second suction chamber which is bounded completely between the suction-ring unit and the surface of the eye, which is not connected to the first suction chamber, and which is capable of being evacuated independently of the latter. The second suction chamber serves for aspiring the suction-ring unit onto the sclera of the eye. For example, the first sealing surface may be formed on the inner edge of an annular projection of the suction-ring unit protruding towards the middle of the ring and directed radially or obliquely relative to the radial, for instance by a ring seal fitted to this annular projection.

In the case of a suction-ring unit mounted properly onto the eye, in the first relative position the first suction chamber may, prior to its evacuation, border completely the underside of the contact glass facing towards the eye. In such an embodiment, in the first relative position prior to evacuation of the first suction chamber the contact glass has no contact with the surface of the eye. As already elucidated, in alternative embodiments it is possible that in the first relative position a comparatively small-area contact already exists between the surface of the eye and the contact glass.

The first suction chamber may reach axially beyond the contact glass in the direction away from the eye.

The suction-ring unit preferentially forms an insertion funnel which opens in the direction axially away from the eye, the interface unit exhibiting a conic section for the purpose of axial insertion into the insertion funnel. The interacting of the insertion funnel and of the conic section permits a precise centring of the interface unit relative to the suction-ring unit. In addition, by virtue of the insertion funnel an axial overall length of the suction-ring unit is obtained that guarantees that the lashes of the eye cannot unintentionally get between the suction-ring unit and the interface unit in troublesome manner.

The insertion funnel and the conic section expediently form interacting second sealing surfaces for sealing the first suction chamber. In this connection the insertion funnel or/and the conic section may possess a full-perimeter groove into which a sealing ring has been inserted. Alternatively, a sealing effect can be achieved also without a separate sealing element, particularly if the insertion funnel and the conic section possess sufficiently smooth surfaces which come to abut one another and in this way can seal the first suction chamber.

The interface unit may include a contact-glass holder which is designed for coupling with the suction-ring unit and which bears the contact glass, as well as an adapter which is separate from the contact-glass holder and capable of being firmly but interchangeably connected to the latter and which is constructed with coupling structures for coupling to focusing optics of a laser device providing the laser radiation. The bipartition of the interface unit into a contact-glass holder and an adapter is advantageous to the extent that after an operation the entire interface unit does not have to be recovered or even thrown away. Instead, the adapter can readily be re-used, where appropriate after prior sterilisation. The contact-glass holder with the contact glass may, on the other hand, be a disposable article. The contact-glass holder and the adapter may, for example, be constructed with thread means which permit a screw connection of the contact-glass holder and the adapter. For the contact-glass holder, use may be made of a conical sleeve body, for example, the sleeve jacket of which consists of solid material or possesses apertures for the purpose of saving weight.

The invention will be elucidated in more detail in the following on the basis of the appended drawings. Represented are:

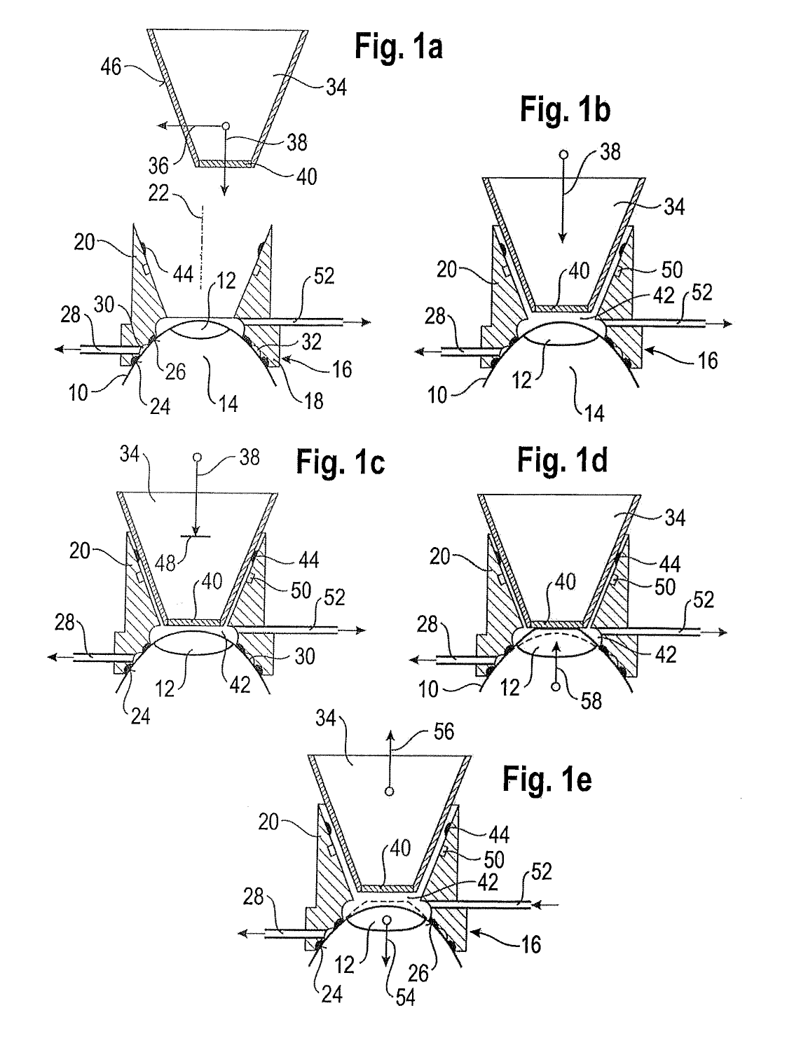

FIGS. 1a-1e schematically, consecutive phases in the course of the coupling and uncoupling of an interface unit to/from a suction-ring unit seated on an eye, according to an exemplary embodiment,

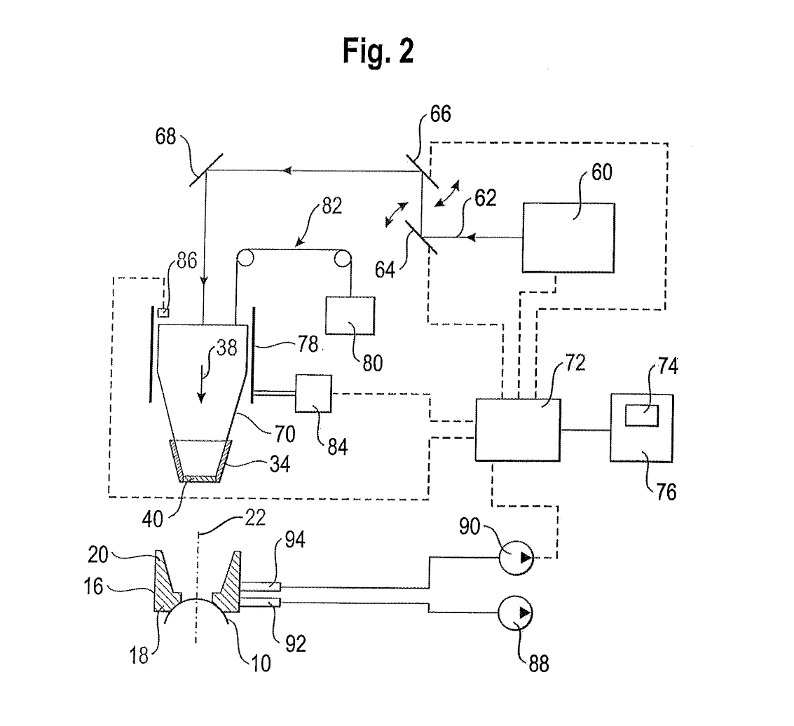

FIG. 2 in greatly schematised manner, an exemplary embodiment of a laser device for introducing incisions into the cornea of an eye,

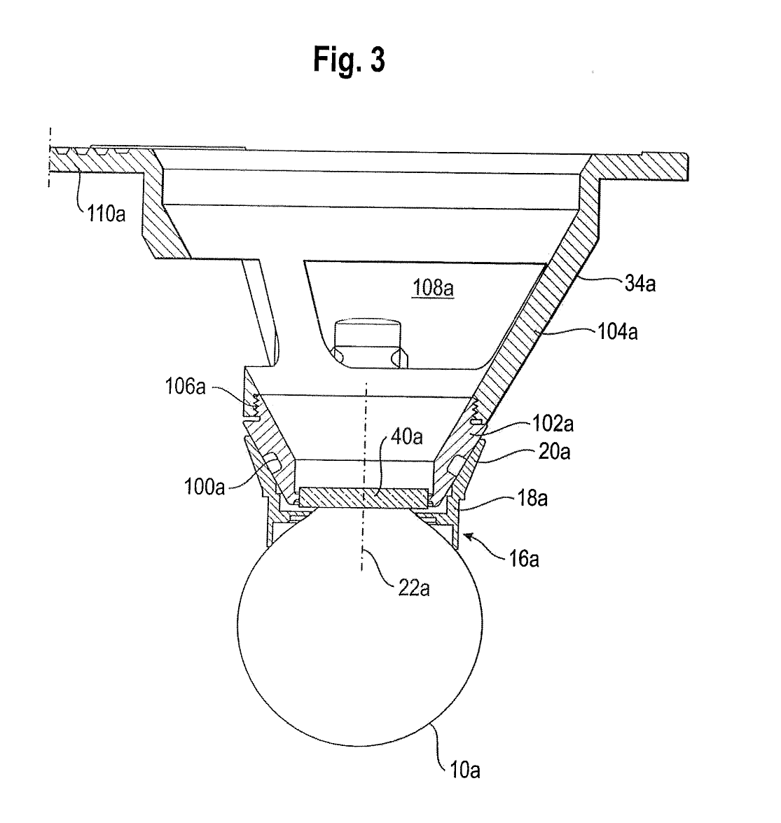

FIG. 3 an axial longitudinal section through a realistically shown exemplary embodiment of an interface unit and of a suction-ring unit in a coupled state,

FIGS. 4a and 4b perspective views of the suction-ring unit according to FIG. 3, in one instance in its entirety and in one instance half cut open, and

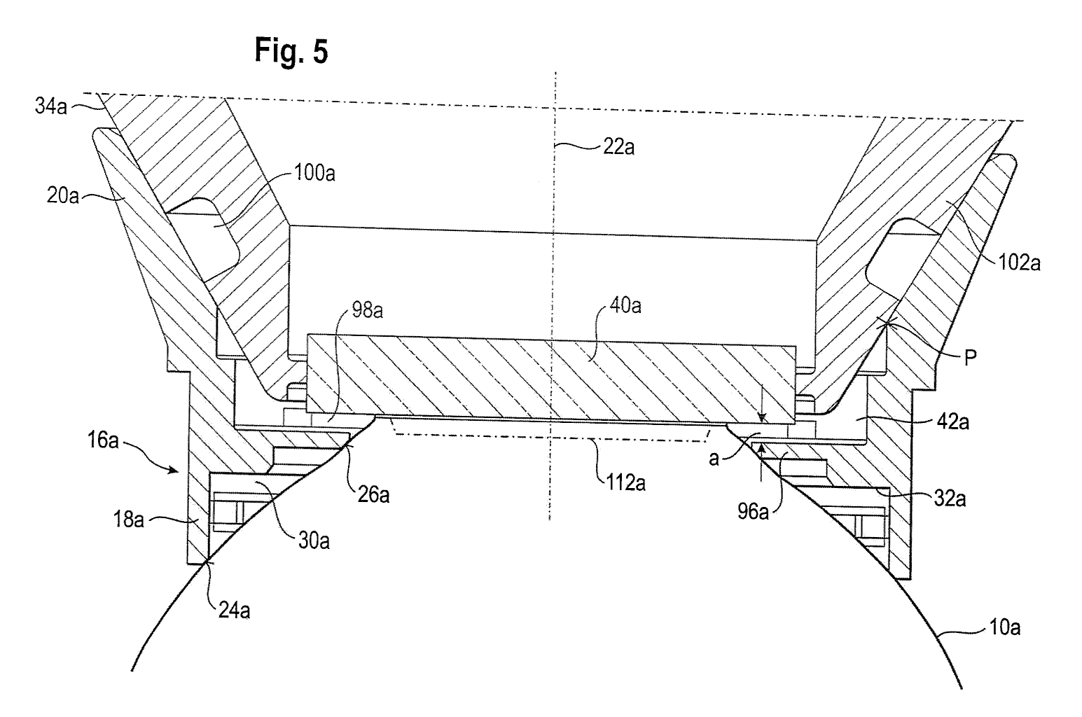

FIG. 5 an enlarged detail from FIG. 3.

In the exemplary embodiment shown in FIGS. 1a-1e the (human) eye to be treated is denoted by 10. The cornea of the eye 10 is shown at 12, whereas the sclera is denoted by 14.

One or more incisions are to be introduced into the cornea 12 of the eye 10 by means of pulsed laser radiation with pulse durations within the femtosecond range. The laser radiation necessary for this is provided by a laser source which is not represented in any detail. For example, the wavelength of the treatment radiation beamed into the eye 10 lies in the low-infrared region. For example, use may be made of an Yb laser radiating at 1030 nm.

Before a start is made with the laser treatment of the eye, the eye 10 firstly has to be coupled to the laser device equipped with the laser source, in order to be able to position the beam focus precisely in the cornea in the z-direction. For this purpose, firstly a suction-ring unit 16 is placed onto the eye 10 in a manner known as such and is fixed to the eye 10 by partial vacuum. The suction-ring unit 16 stabilises and fixes the eye 10. It exhibits a lower part 18, forming the actual suction ring 18, an insertion funnel 20 connected to the lower part 18 and produced integrally with the latter, and also a ring axis 22. The lower part 18 forms two full-perimeter sealing surfaces 24, 26 in the form of a ring, each intended for abutment against the sclera 14, which bound between themselves a full-perimeter suction chamber 30 in the form of a ring, connected to an evacuation channel 28. The sealing surfaces 24, 26 may, for example, each be formed by a separate sealing element fitted to the lower part 18. For the purpose of forming the suction chamber 30, on the inner-periphery shell of the lower part 18 facing towards the eye a corresponding annular groove—denoted by 32—is formed. The suction chamber 30 is bounded exclusively between the suction-ring unit 16 and the sclera 14. As a result of evacuation of the suction chamber 30, the suction-ring unit 16 is firmly attached to the eye 10 by suction. For this purpose, the evacuation channel 28 is connected to a source of partial vacuum, not represented in any detail, in the form of an evacuation pump.

A mechanical companion part to the suction-ring unit 16 which is fixed on the eye 10 in such a way is an interface unit, generally denoted by 34, which, in a manner not represented in any detail, is capable of being firmly but releasably coupled with focusing optics of the aforementioned laser device. The interface unit 34 is capable of being displaced, together with the focusing optics, relative to the patient and to the suction-ring unit 16 fastened to him/her along a horizontal direction indicated by a horizontal arrow 36 and along a vertical direction indicated by a vertical arrow 38. The capacity for displacement of the interface unit 34 may be brought about at least partly by motorised means, for example by means of an electromotive drive. An at least partly manual capacity for movement of the interface unit 34 relative to the suction-ring unit 16 is also conceivable.

Overall, the interface unit 34 is of conical design, being formed at its wider cone end (at the top in FIGS. 1a-1e) for coupling with the focusing optics, and bearing at its narrower cone end a contact glass 40 taking the form, in the exemplary case that is shown, of a plane-parallel applanation lens.

In a first phase of the procedure for coupling the eye 10 to the laser device the interface unit 34 is moved relative to the suction-ring unit 16 in the arrow direction 36 into a position in which it is located axially above the insertion funnel 20, so that the interface unit 34 can subsequently enter the insertion funnel 20 by being axially lowered. The phase of the entering of the interface unit 34 into the insertion funnel 20 of the suction-ring unit 16 is represented in FIG. 1b. In the course of the lowering of the interface unit 34, the applanation lens 40 approaches the eye 10; at the same time, the radial air gap between the insertion funnel 20 and the interface unit 34 becomes smaller. The conically extending inner-periphery shell of the insertion funnel 20 and the equally conically extending outer-periphery shell of the interface unit 34 bear or form in each case a sealing surface. These interacting sealing surfaces come to abut one another in the course of the further coupling procedure and in this state seal a further suction chamber 42. The further suction chamber 42 is formed between the suction-ring unit 16, the interface unit 34 and the surface of the eye 10. The sealing surface 26 serves as lower boundary of the suction chamber 42 and accordingly seals both suction chambers 30, 42 at the same time. It will be understood that alternatively a further annular sealing surface, separate from the sealing surface 26, may be formed on the suction-ring unit 16, said annular sealing surface serving for sealing the suction chamber 42.

The sealing surfaces that are effective between the suction-ring unit 16 and the interface unit 34 are, in the exemplary case that is shown, formed by a ring seal 44 fitted to the insertion funnel 20 and by the part of the outer-periphery shell of the interface unit 34 situated opposite this ring seal 44 in the entered state. This part of the outer-periphery shell of the interface unit 34 acting as a sealing surface is denoted by 46 in FIG. 1a. The ring seal 44 may, for example, be a lip seal or an O-ring. It will be understood that alternatively such a ring seal may be provided on the interface unit 34. It is furthermore conceivable to dispense with a separate sealing element, provided that the outer surface of the interface unit 34 and the inner surface of the insertion funnel 20 are sufficiently smooth and come to be located alongside one another sufficiently closely.

The lowering of the interface unit 34 in the arrow direction 38 (corresponding to the axial direction of the suction-ring unit 16) stops at a predetermined axial relative position in which the interface unit 34 still has a certain axial spacing from the suction-ring unit 16—that is to say, has not yet entered the insertion funnel 20 to maximal depth. The stop of the lowering movement is indicated schematically in FIG. 1c by a transverse line 48. In the stop position of the interface unit 34 there is, on the one hand, still a radial gap between the insertion funnel 20 and the interface unit 34; on the other hand, no significant levelling of the eye by the applanation plate 40 has yet taken place. For example, the size of the air gap between insertion funnel 20 and interface unit 34 when the stop position is attained amounts to less than 0.5 mm, right down to no more than 0.1 mm.

In the exemplary case of FIG. 1c that is shown, in the stop position of the interface unit 34 there is no contact at all between the applanation plate 40 and the eye 10; instead, the applanation plate 40 is located at a short axial spacing above the cornea 12 of the eye 10. But alternatively in the stop position there may also be a slight contact between the applanation plate 40 and the eye; the surface of the region of contact is then, of course, considerably smaller than the applanation region required for the later treatment. For example, the levelling region of the eye that is striven for may exhibit a diameter between about 10 mm and 11 mm. To the extent that in the stop position according to FIG. 1c a contact is present at all between the applanation plate 40 and the eye 10, the diameter of the region of contact will, on the other hand, preferentially amount to at most only a few millimetres, for example only about 2 mm or 3 mm. In each case a contact possibly existing in the stop position between applanation plate 40 and eye 10 is so weak that no significant increase in the intraocular pressure is brought about by the contact.

The stop position of the interface unit 40 is detected, in the exemplary case that is shown, by a sensor element 50 arranged on the suction-ring unit 16, more precisely on the insertion funnel 20, which may be, for example, a Hall-effect sensor taking the form of a proximity sensor. The sensor element 50 is suitably positioned in order to emit an appropriate signal when the interface unit 34 attains the predetermined stop position.

In the stop position the suction chamber 42 extends into the annular gap still existing between the insertion funnel 20 and the interface unit 34. The sealing element 44 may already be in sealing abutment on the conical outer-periphery shell of the interface unit 34. But it is also possible that in the stop position there is still no complete sealing of the suction chamber 42. Depending on whether a contact between the applanation plate 40 and the eye 10 is already present or not in the stop position, the suction chamber 42 either extends completely through between the applanation plate 40 and the eye 10 or surrounds the existing region of contact.

The suction chamber 42 is connected to a further evacuation channel 52 which, like the evacuation channel 28, is formed on the suction-ring unit 16 and likewise is capable of being connected to an evacuation pump (not represented in any detail) serving as source of partial vacuum. Via the two evacuation channels 28, 52 the two suction chambers 30, 42 can be evacuated independently of one another. For this purpose, two separate evacuation pumps, capable of being driven independently of one another, may be provided. Alternatively it is conceivable to provide a single evacuation pump and to supply the two suction chambers 30, 42 individually with partial vacuum by means of suitably controllable valve means.

Starting from the stop position according to FIG. 1c, by way of next action the suction chamber 42 is evacuated. The increasing partial vacuum in the suction chamber 42 brings about an aspiration of the surface of the eye onto the underside (facing towards the eye) of the applanation plate 40. At the same time, the interface unit 34 is drawn deeper into the insertion funnel 20 of the suction-ring unit 16 by the suction effect until it finally attains a maximally deep entry position. This further lowering of the interface unit 34 is brought about solely by the suction effect of the partial vacuum prevailing in the suction chamber 42; no motorised or manual force support any longer takes place in this phase of the coupling procedure. The extent of this further lowering movement of the interface unit 34 is comparatively slight; for example, due to suction force the interface unit 34 is sucked deeper into the insertion funnel 20 by only a few tenths of a millimetre.

The strength of the partial vacuum generated in the suction chamber 42 is chosen to be sufficiently high in order to level the eye 10 in the region desired for the treatment, i.e. to flatten it by aspiration onto the plate 40. In the process, the increase in contact brought about by the drawing of the interface unit 40 into the insertion funnel 20 is in each case small and preferentially even negligible in comparison with the enlargement of the region of contact brought about as a consequence of the aspiration of the cornea. In order to mention exemplary numerical values, in this way a partial vacuum between 200 mm Hg and 600 mm Hg, for example, can be generated in the suction chamber 42, preferentially between 400 mm Hg and 500 mm Hg.

The flattened state in which the suction chamber 42 is evacuated to the desired level is shown in FIG. 1d. In this state, by beaming laser radiation from above through the applanation plate 40 the desired treatment of the eye 10 can be effected, for example the generation of a flap within the scope of an fs LASIK treatment.

Should there be problems in the state according to FIG. 1d, possibly because the patient reacts with panic, a rapid uncoupling of the interface unit 34 from the suction-ring unit 16 can be obtained by aeration of the suction chamber 42. This situation is represented in FIG. 1e. A downward-directed arrow 54 illustrates the instantaneous detachment of the eye 10 from the applanation plate 40. On account of the absent suction effect, the interface unit 34 can then also escape axially upwards out of the insertion funnel 20. This is illustrated by an upward-directed arrow 56.

In comparison to FIG. 1e, in FIG. 1d the suction effect exerted on the surface of the eye is illustrated by an upward arrow 58 directed towards the applanation plate 40.

In a process as elucidated above, it is possible to keep an increase in the intraocular pressure brought about by the coupling of the interface unit 34 to the suction-ring unit 16, i.e. by the levelling of the cornea, not only small in absolute terms but also small in comparison to an increase in pressure as a consequence of the aspirating of the suction-ring unit 16 onto the sclera. In order to give a numerical example, the fixation of the suction-ring unit 16 to the eye may already bring about an increase in the intraocular pressure by about 60 mm Hg to 100 mm Hg. For comparison, customary intraocular pressures of the human eye without contact amount, as a rule, to between about 15 mm Hg and 20 mm Hg. If in the stop position of the interface unit 34 prior to the start of the evacuation of the suction chamber 42 a contact is already present between eye and applanation plate 40, the rise in pressure brought about in the eye by this means is preferentially less than 10 mm Hg. Such a small rise in pressure can readily be guaranteed with a contact having a diameter of 2 mm to 3 mm. In each case a contact existing in the stop position prior to the start of the evacuation should preferentially not lead to a rise in pressure by more than 20 mm Hg. By virtue of the subsequent evacuating of the suction chamber 42 and the aspiration of the eye onto the applanation plate, a further slight rise in pressure in the eye may occur, which, however, should amount, as a rule, to no more than approximately 20 mm Hg, depending on the partial vacuum in the suction chamber 42.

FIG. 2 shows schematically components of a laser device with which the process described previously on the basis of FIGS. 1a-1e can be implemented. Components that are identical to, or that act identically to, those in FIGS. 1a-1e are denoted in this case by identical reference symbols. With a view to avoiding repetition, reference is made to the preceding remarks relating to these components.

The laser device according to FIG. 2 includes a laser source 60 for pulsed laser radiation with pulse durations within the femtosecond range. The laser beam—denoted by 62—emitted by the laser source 60 reaches, via a deflecting device (scanner) formed here by two controllable deflecting mirrors 64, 66, a deviating mirror 68, from which the laser beam 62 reaches focusing optics 70. At the distal end, i.e. the end close to the eye, of the focusing optics 70 the interface unit 34 is releasably coupled. The deflecting mirrors 64, 66 are each arranged so as to be tiltable and permit a deflection of the laser beam 62 in an x-y plane which is normal to the longitudinal direction of the beam (z-direction). They are controlled by an electronic control device 72 in accordance with an incision profile given by the shape and position of the desired incision. The incision profile is embodied in a control program 74 which is saved in a memory 76 which can be accessed by the control device 72. For the purpose of z-relocation of the beam focus, either the focusing optics 70 or at least one lens contained therein may be adjustable in the longitudinal direction of the beam, under the control of the control device 72. Alternatively it is possible to arrange a lens of beam-expanding optics, not represented in any detail in FIG. 2, arranged between the laser source 60 and the deflecting mirrors 64, 66 so as to be relocatable in the longitudinal direction of the beam, in particular an input-side diverging lens of such beam-expanding optics.

The focusing optics 70 are suspended in weight-compensated manner on a mounting 78. The mounting 78 is indicated in FIG. 2 in greatly schematic manner by two vertical dashes drawn on either side of the focusing optics 70. The weight compensation of the focusing optics 70 is indicated schematically by a counterweight 80 which is connected to the focusing optics 70 via a rope/pulley arrangement 82 and which exerts a counterforce on the focusing optics 70 compensating the weight of said focusing optics. A rope/pulley arrangement is, of course, only one example of the fastening of a counterweight to the focusing optics. Alternatively, use could be made of a lever system, for example. Another possible configuration is shown in U.S. Pat. No. 5,336,215, where a spring system is used for the purpose of suspending focusing optics.

The focusing optics 70 are capable of being lowered, together with the mounting 78, in the vertical direction into the insertion funnel 20 of the suction-ring unit 16 seated on the eye 10 by a motorised, preferentially electromotive, drive unit 84, as indicated by the direction arrow 38. In this case the focusing optics 70 are not rigidly connected to the mounting 78 but possess in relation to the mounting 78 a certain capacity for displacement upwards contrary to the lowering direction 38. On account of the weight compensation of the focusing optics 70, a displacement of the same in relation to the mounting is already possible by virtue of an extremely small application of force. The moment at which the applanation plate 40 comes into contact with the eye and experiences a counterpressure from the eye may therefore already result in a displacement of the focusing optics 70 relative to the mounting 78. This displacement is detected by means of a limit switch 86 (alternatively, for example, a counterforce switch) which is fitted in stationary manner relative to the mounting 78 and which provides its switching signal to the control device 72. The switching of the limit switch 86 consequently signals to the control device 72 the attaining of the predetermined relative position between interface unit 34 and suction-ring unit 16 (e.g. touching of the insertion funnel 20 by the interface unit 34), in which the further motorised lowering movement of the focusing optics 70 has to be stopped. Accordingly, when the switching signal is received from the limit switch 86 the control device 72 controls the drive unit 84 in the sense of an operational stop. The previous lowering of the focusing optics 70 by the drive unit 84 can likewise be controlled by the control device 72 in accordance with the control program 74; alternatively it is conceivable that the operator initialises the lowering movement manually by means of a joystick connected to the control device 72, in which case when the aforementioned predetermined relative position between interface unit 34 and suction-ring unit 16 is attained the control device 72 cancels the precedence of the joystick and stops the operation of the drive unit 84 automatically.

On account of the presence of the limit switch 86, in this case a sensor fitted to components 16 or/and 34 may be dispensed with.

The laser device according to FIG. 2 furthermore includes two evacuation pumps 88, 90 which via suitable hose lines are connected to a pipe nipple 92 and 94, respectively, formed on the suction-ring unit 16. Evacuation channel 28 according to FIGS. 1a-1e leads into pipe nipple 92; evacuation pump 88 consequently serves for evacuating suction chamber 30. On the other hand, evacuation channel 52 leads into pipe nipple 94, on account of which evacuation pump 90 serves for evacuating suction chamber 42 shown in FIGS. 1a-1e. In the exemplary case according to FIG. 2 which is represented, at least evacuation pump 90 is capable of being controlled by the control device 72, namely in response to the switching of the limit switch 86. This means an automatic switching-on of pump 90 takes place as soon as the interface unit 34 has attained its predetermined relative position in relation to the suction-ring unit 16. The switching of the limit switch 86 can incidentally be triggered not only by a displacement of the focusing optics 70 as a consequence of a contact with the eye. For example, the counterpressure required for this can also be generated by a ring seal (e.g. lip seal) fitted to the insertion funnel 20 or to the interface unit 34, for instance by the ring seal 44 shown in FIGS. 1a-1e.

Within the scope of a fully automatic operation of the laser device, evacuation pump 88 may also be capable of being controlled by the control device 72. But it is just as conceivable that at least the firm attachment of the suction-ring unit 16 onto the eye 10 by suction is brought about by manual actuation of evacuation pump 88 by the operating physician. Such a capacity for manual switching on and off is, incidentally, also not excluded for pump 90. Particularly in the case of complications, at least an ability to switch off evacuation pump 90 manually should be possible for the operating physician.

In connection with the elucidation of the exemplary embodiment shown in FIGS. 3-5, recourse will again be had to reference symbols identical to those used previously, to the extent that it is a question of components that are identical, or that act identically, to those in the preceding exemplary embodiments. For the purpose of differentiation, however, this time a lower-case letter is appended to the reference symbol in question. Unless otherwise stated in the following, reference is made to what was stated above for an elucidation of the aforementioned identical or identically acting components.

In the exemplary embodiment shown in FIGS. 3-5 the lower part 18a of the suction-ring unit 16a possesses a washer-shaped sealing and separating plate 96a projecting radially towards the centre of the ring, which on its inner edge directly forms the sealing surface 26a or bears a sealing ring forming the sealing surface 26a. The sealing and separating plate 96a separates the two suction chambers 30a, 42a from one another. It can be discerned that the applanation plate 40a reaches radially beyond the sealing and separating plate 96a and that the suction chamber 42a extends into the region between the applanation plate 40a and the sealing and separating plate 96a. In the state according to FIGS. 3 and 5, in which the interface unit 34a is properly coupled to the suction-ring unit 16a and the suction chamber 42a is evacuated, the axial spacing between the underside of the applanation plate 40a, facing towards the eye, and the upper side of the sealing and separating plate 96a amounts, for example, to about 0.7 mm. Overall, for this spacing a dimension has proved to be expedient that should be no less than 0.4 mm, in order to avoid undesirable pressure influences of the applanation plate on the eye, but at the same time no greater than 1.2 mm, in order to be able to aspirate the eye onto the applanation plate 40a in a sufficiently large region of contact and thereby to be able to level it. For the purpose of illustration, the stated axial spacing is identified by a in FIG. 5. Depending on the configuration of the applanation plate 40a (it could also be stepped instead of completely flat), the stated spacing may, at least in the marginal region of the plate 40a, under certain circumstances also be small, right down to 0.1 mm.

As already elucidated, the evacuation of the suction chamber 42a may have the result that the interface unit 34a is sucked deeper into the insertion funnel 20a of the suction-ring unit 16a by a comparatively small axial dimension. At the start of the evacuation, the spacing a is therefore greater by this dimension than in the evacuated state according to FIGS. 3 and 5.

It can further be discerned in FIG. 5 that the suction chamber 42a extends laterally on the outside past the lower cone end of the interface unit 34a in the axially upward direction into a region that is situated axially above the applanation plate 40a. This axial point is denoted by P in FIG. 5. On the other side of point P, i.e. in the direction towards greater aperture widths of the insertion funnel 20a, there is moulded into the conical outer-periphery shell of the interface unit 34a a full-perimeter annular groove 100a which serves for receiving a sealing element, not represented here in any detail, which interacts with the inner funnel surface of the insertion funnel 20a for the purpose of sealing the suction chamber 42a.

In FIG. 3 it can further be discerned that the interface unit 34a may be of bipartite construction and includes a conic sleeve 102a retaining the applanation plate 40a, as well as an adapter cone 104a which is releasably connected to the conic sleeve 102a, for example by means of a screw connection, as indicated at 106a. Solely the conic sleeve 102a enters the insertion funnel 20a; the adapter cone 104a always remains axially outside the suction-ring unit 16a. In the exemplary case that is shown, the wall of the adapter cone 104a is penetrated by several apertures 108a, in order to is save weight. The conic sleeve 102a, on the other hand, takes the form of a solid-shell body. At the proximal end, i.e. the end remote from the eye, the adapter cone 104a is constructed for releasable coupling (for example, in the form of an axial clamp coupling) with the focusing optics of the laser device. In the exemplary case that is shown, for this purpose it exhibits a radially projecting annular collar 110a serving as clamping flange.

It will be understood that alternatively a one-piece or even more than two-piece configuration of the interface unit is conceivable.

A dash-dotted line 112a in FIG. 5 illustrates an exemplary incision shape such as can be made in the cornea of the eye 10a by means of laser pulses suitably controlled in space and time. It will be understood that a large number of other incision shapes are possible, depending on the desired type of treatment.

1. Process for coupling a mechanical interface unit of a laser device to a stabilising component retained on a biological tissue by suction force, comprising the following steps:
relative approximating of the interface unit to the stabilising component as far as a first relative position,
in the first relative position, evacuating a suction chamber formed between the interface unit, the stabilising component and the surface of the tissue, in order thereby to establish a contact between the tissue and a tissue-abutment surface of the interface unit, or to enlarge a region of existing contact.

2. Process according to claim 1, characterised in that the evacuation of the suction chamber brings about an enlargement of the region of contact between the tissue and the tissue-abutment surface to at least one and a half times, preferentially to at least double, in particular to a multiple.

3. Process according to claim 1, characterised in that the region of contact between the tissue and the tissue-abutment surface is approximately circular-disc-shaped and the evacuation of the suction chamber brings about an enlargement of the diameter of the region of contact by at least 30 percent, preferentially by at least 50 percent, still more preferably by at least 70 percent, and most preferably by at least 90 percent.

4. Process according to claim 1, characterised in that the relative approximation of the interface unit to the stabilising component is effected in a direction along which a further relative approximation of the interface unit and of the stabilising component beyond the first relative position is possible, in particular as far as a state of reciprocal impact.

5. Process according to claim 1, characterised in that the evacuation of the suction chamber brings about a relative movement of the interface unit and of the stabilising component into a second relative position in which they are more greatly approximated than in the first relative position.

6. Process according to claim 5, characterised in that the path of relative movement of the interface unit and of the stabilising component between the first and the second relative position amounts to no more than 1 mm.

7. Process according to claim 5, characterised in that a proportion of the enlargement of the region of contact between the tissue and the tissue-abutment surface to be ascribed to the relative approximation of the interface unit and of the stabilising component from the first into the second relative position is small, in particular negligibly small in comparison to a proportion to be ascribed to the aspirating of the tissue onto the tissue-abutment surface.

8. Process according to claim 1, characterised in that the attaining of the first relative position is detected by means of sensorics and the evacuation of the suction chamber is brought about automatically, in particular in program-controlled manner, in response to the detection of the first relative position.

9. Process according to claim 1, characterised in that the relative approximation of the interface unit to the stabilising component is brought about by actuation of a motorised drive device and in that the operation of the drive device is stopped automatically, in particular in program-controlled manner, in response to a sensoric detection of the first relative position.

10. Apparatus for cutting a tissue part of an eye by means of focused laser radiation, in particular for the purpose of implementing the process according to one of the preceding claims, comprising
a suction-ring unit to be placed onto the eye, with a ring axis,
a mechanical interface unit which is separate from the suction-ring unit and capable of being moved along the ring axis in coupling contact with the latter, with a contact glass for shaping the surface of the eye,
pumping means for evacuating a first suction chamber bounded between the suction-ring unit, the interface unit and the surface of the eye,
sensorics for detecting a predetermined first axial relative position of the interface unit and of the suction-ring unit,
a control device connected to the sensorics and to the pumping means, which has been set up to bring about an evacuation of the first suction chamber in response to the detection of the first relative position by the sensorics, in particular in order thereby to establish a contact between the eye and a shaping abutment surface of the contact glass, or to enlarge a region of existing contact.

11. Apparatus according to claim 10, characterised in that the suction-ring unit forms a full-perimeter first sealing surface in the form of a ring, with which it is capable of being applied to the surface of the eye for the purpose of sealing the first suction chamber, whereby the evacuation of the first suction chamber brings about an abutment of the eye against the contact glass in a region that corresponds approximately to the area surrounded by the first sealing surface.

12. Apparatus according to claim 11, characterised in that the first sealing surface is formed on the inner edge of a washer body formed on the suction-ring unit and projecting towards the eye, which separates the first suction chamber from a second suction chamber bounded completely between the suction-ring unit and the surface of the eye, which is not connected to the first suction chamber, and which is capable of being evacuated independently of said first suction chamber.

13. Apparatus according to claim 10, characterised in that in the case of a suction-ring unit properly placed onto the eye in the first relative position the first suction chamber completely borders, prior to its evacuation, the underside of the contact glass facing towards the eye.

14. Apparatus according to claim 10, characterised in that the first suction chamber reaches axially beyond the contact glass in the direction away from the eye.

15. Apparatus according to claim 10, characterised in that the suction-ring unit forms an insertion funnel which opens in the direction axially away from the eye and the interface unit exhibits a conic section for axial insertion into the insertion funnel.

16. Apparatus according to claim 15, characterised in that the insertion funnel and the conic section form interacting second sealing surfaces for sealing the first suction chamber.

17. Apparatus according to claim 15, characterised in that the insertion funnel or/and the conic section possess(es) a full-perimeter groove into which a sealing ring has been inserted.

18. Apparatus according to claim 10, characterised in that the interface unit includes a contact-glass holder designed for mechanical coupling with the suction-ring unit and bearing the contact glass, as well as an adapter which is separate from the contact-glass holder and capable of being firmly but interchangeably connected to the latter and which is constructed with coupling structures for coupling to focusing optics of a laser device providing the laser radiation.

19. Apparatus according to claim 18, characterised in that the contact-glass holder and the adapter are constructed with thread means which permit a screw connection of the contact-glass holder and the adapter.

20. Apparatus according to claim 18, characterised in that that the contact-glass holder is designed in the form of a cone sleeve.

21. Apparatus according to claim 10, characterised in that at least the side of the contact glass facing towards the eye is constructed as a plane surface.
